(19) United States
(12) Patent Application Publication
(10) Pub. No.: US 2016/0237114 A1
(43) Pub. Date: Aug. 18, 2016

(54) PROTEIN AFFINITY PURIFICATION RESINS WITH TAGGED PROTEIN

(71) Applicant: PURDUE RESEARCH FOUNDATION, West Lafayette, IN (US)

(72) Inventors: Tamara Lea Kinzer-Ursem, West Lafayette, IN (US); Chethana Kulkarni, Beverly, MA (US); Julia Grace Fraseur, West Lafayette, IN (US)

(73) Assignee: PURDUE RESEARCH FOUNDATION, West Lafayette, IN (US)

(21) Appl. No.: 15/044,393

(22) Filed: Feb. 16, 2016

Related U.S. Application Data
(60) Provisional application No. 62/116,637, filed on Feb. 16, 2015.

(51) Int. Cl. C07K 1/22; C07K 14/47

(52) U.S. Cl. CPC C07K 1/22; C07K 14/4728

(57) ABSTRACT

Disclosed herein are protein enrichment substrates and methods for making the same that can be used to enrich proteins of interest. Also disclosed herein are methods for protein purification using these protein enrichment substrates.

### CROSS-REFERENCE TO RELATED APPLICATIONS

The present U.S. patent application is related to and claims the priority benefit of U.S. Provisional Patent Application Ser. No. 62/116,637, filed Feb. 16, 2015, the contents of which is hereby incorporated by reference in its entirety into this disclosure.

### TECHNICAL FIELD

The present disclosure generally relates to protein engineering, and in particular to a method for making protein functionalized materials that can be used to enrich proteins of interest.

### BACKGROUND

This section introduces aspects that may help facilitate a better understanding of the disclosure. Accordingly, these statements are to be read in this light and are not to be understood as admissions about what is or is not prior art.

As an example of methods that are regularly used to study protein function we present information about one protein, calmodulin (CaM) that is a widely studied calcium binding protein with many protein binding partners. Calcium (Ca²⁺)-binding proteins play an important role in mediating intracellular and intercellular communication, as well as in activating a number of protein-protein interactions and corresponding biological processes. Ca²⁺-sensor proteins, which comprise one of two classes of Ca²⁺-binding proteins, transmit information about Ca²⁺ flux duration and magnitude to downstream protein signaling cascades. A widely studied member of the Ca²⁺-binding protein family is calmodulin (CaM), a small (16 kDa) and ubiquitous Ca²⁺ sensor that binds and activates more than 100 other proteins. CaM is highly conserved across evolution: remarkably, its amino acid sequence is identical in all vertebrates. CaM is also part of a larger family of proteins that contain EF-hand motifs shared with prokaryotes. In addition, CaM is found in nearly every tissue of the human body; indeed, many cellular and physiological processes, ranging from cell proliferation, vesicle release, and apoptosis to neuronal signaling and muscle contraction, depend on Ca²⁺/CaM-activated proteins. While there are a number of CaM-binding proteins with well-characterized functions, especially in neuronal and muscle tissues, there is also a body of work describing Ca²⁺/CaM-activated proteins whose biochemical functions and physiological roles are not well-described. Thus, there remains a need for streamlined methods to enable detailed studies of CaM, CaM-binding proteins, and the interactions between these partners. This is also the case for many other proteins and protein families.

Similar to many other proteins, previous studies of CaM biochemical function have utilized established protein labeling strategies to quantitatively measure the binding of Ca²⁺ to CaM, and subsequent binding of CaM to downstream proteins. Many of these studies have taken advantage of the fact that CaM is a small protein with relatively few reactive amino acids; vertebrate CaM, in particular, possesses no Cys residues. Thus, selective labeling can be achieved at Cys sites that are introduced in mutated vertebrate CaM or that are present in non-vertebrate CaM. Through the precise introduction into CaM of small fluorescent probes that are sensitive to their immediate micro-environment, researchers have elegantly elucidated the biochemical mechanisms underpinning the binding steps in which CaM participates. However, a survey of these extrinsic labeling techniques reveals three main drawbacks: they generally require purification of protein both before and after labeling; they are non-selective either in amino acid site or side-chain chemistry; and most significantly, they often lead to decreases in the ability of protein to bind and activate its target proteins. There is therefore also an unmet need for a method for making protein-functionalized materials that can be used to enrich proteins of interest for further study.

### SUMMARY

In one aspect, a protein enrichment substrate is presented. The protein enrichment substrate is prepared by a process that includes incubating a starting substrate with a mixture to produce a functionalized substrate to thereby make the protein enrichment substrate. The mixture can include a bioorthogonally tagged protein for selective reaction with the substrate.

In another aspect, a method for making an protein enrichment substrate is presented. The method includes incubating a substrate with a mixture to produce a functionalized substrate to thereby make the protein enrichment substrate.

In yet another aspect, a method of protein enrichment is presented, which includes the steps of incubating a protein enrichment substrate with a buffer to produce an equilibrated mixture of substrate and buffer, further incubating the equilibrated substrate with a crude mixture containing a binding protein to produce a second mixture of functionalized substrate and crude mixture to produce a protein bound substrate, washing the protein bound substrate to produce a washed substrate, and eluting the binding protein from the substrate with another buffer to thereby result in an enriched protein. The protein enrichment substrate is prepared by a process that includes the steps of incubating a starting substrate with a mixture to produce a functionalized substrate to thereby make the protein enrichment substrate, wherein the mixture can include a bioorthogonally tagged protein for selective reaction with the substrate.

### BRIEF DESCRIPTION OF THE FIGURES

FIGS. 1A-1F are schematic representations of the reagents and proteins described herein. FIGS. 1A-1C are reagents used in the study described herein: 12-azidododecanoic acid (ADA, FIG. 1A), azadibenzocyclooctyne-amine (ADIBO-NH₂, FIG. 1B), and azadibenzocyclooctyne-(PEG)₄-amine (ADIBO-PEG-NH₂, FIG. 1C). FIG. 1D shows Ca²⁺-bound CaM, PDB ID: 1CLL. FIG. 1E shows CaM bound to a peptide derived from the regulatory domain of calcineurin, PDB ID: 4Q5U. In FIGS. 1D and 1E, arrows point to the amino-terminus of CaM. FIG. 1F shows how CaM was engineered to display NMT recognition sequences (RS1 and RS2) and linker regions (L1 and L2) to produce a small library of enzymatically labeled, ADA-tagged proteins.

FIGS. 2A-2F relate to the SDS-PAGE analysis of phenyl sepharose purification fractions for CaM proteins. Purification fractions were collected from expression cultures of WT CaM (FIG. 2A), ADA-RS1-CaM (FIG. 2B), ADA-RS1-L1-CaM (FIG. 2C), ADA-RS2-CaM (FIG. 2D), ADA-RS2-L1-CaM (FIG. 2E), and ADA-RS2-L2-CaM (FIG. 2F). All CaM proteins except WT CaM were expressed in the presence of ADA for N-terminal labeling by NMT. *=protein marker, CL=clarified lysate, FT=flow-through, W=wash, E=elution, B1=0.05 mg/mL BSA, B2=0.1 mg/mL BSA.

FIG. 3 is a plot showing the ability of RS2-L2-CaM to activate the CaN phosphatase is not affected by the presence or absence of the ADA label, as evidenced by the similarity in activity data for ADA-RS2-L2-CaM and RS2-L2-CaM. n=4. Error bars correspond to SDs.

FIGS. 4A-4E show preparation and use of protein affinity resin. FIG. 4A is a schematic representation showing preparation of protein affinity resin directly from cell lysate: Step (1) ADIBO-functionalized resin is incubated with clarified lysate containing ADA-tagged protein, which reacts covalently with the ADIBO-resin via the strain-promoted azide-alkyne cycloaddition; Step (2) Protein affinity resin is washed and equilibrated with binding buffer; Step (3) Protein affinity resin is incubated with clarified lysate containing the protein to be purified (binding protein); Step (4) The protein affinity resin is washed, and the binding protein is eluted. FIGS. 4B-4E are representative results of CaN purification from lysate with the following CaM affinity resins: FIG. 4B shows CnBr-activated resin reacted with pure WT CaM, FIG. 4C shows NHS-activated resin reacted with pure WT CaM, FIG. 4D shows ADIBO-PEG-resin prepared by reacting NHS-activated resin with ADIBO-PEG-NH2 and incubated with lysate containing ADA-RS1-CaM, FIG. 4E shows ADIBO-PEG-resin incubated with lysate containing WT CaM. *=protein marker, CL=clarified lysate, FT=flow-through, W=wash, and E=elution. A larger quantity of CaN is recovered with resin prepared from ADA-RS1-CaM lysate than with either resin prepared from pure WT CaM.

FIG. 5 relates to Quantitative Western blot analysis of cell lysates containing ADA-RS1-CaM. Western blot: The indicated amounts of clarified cell lysate from a bacterial expression culture of ADA-RS1-CaM were analyzed alongside known quantities of pure CaM. The table within FIG. 5 shows the amount of ADA-RS1-CaM in each lysate sample was calculated based on a standard curve prepared from the pure CaM band intensities.

FIGS. 6A and 6B relate to purification of CaN using CaM-affinity resins. Representative results of CaN purification from lysate with (FIG. 6A) ADIBO-resin prepared by reacting NHS-activated resin with ADIBO-NH2 and incubated with lysate containing ADA-RS1-CaM, or (FIG. 6B) ADIBO-resin incubated with lysate containing WT CaM. *=protein marker, CL=clarified lysate, FT=flow-through, W=wash, and E=elution. Effective purification of CaN is achieved using ADIBO-resin that has been incubated with ADA-RS1-CaM, but not with WT CaM, suggesting that only ADA-RS1-CaM is covalently captured by ADIBO-resin.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In response to the unmet need, in one aspect, a protein enrichment substrate is presented, which is prepared by a process that includes incubating a starting substrate with a mixture to produce a functionalized substrate to thereby make the protein enrichment substrate. The mixture can be a bioorthogonally tagged protein for selective reaction with the substrate. The starting substrate is configured to be reactive to bioorthogonally labeled proteins. The mixture can be a crude mixture. The mixture can also be a purified protein mixture. The starting substrate can be any one of or a combination of magnetic beads, polystyrene beads, nanoparticles, a chromatography resin, silica oxide, and a polymer. It should be noted that in a preferred embodiment, the nanoparticles can be in a sub-100 nanometer range and can include any material in nanoparticle form (including but not limited to gold, silver, and quantum dots). The polymer can be a polymer matrix.

In another embodiment, a method for making a protein enrichment substrate is presented, which includes the step of incubating a substrate with a mixture to produce a functionalized substrate to thereby make the protein enrichment substrate. In one embodiment, the mixture can be a crude mixture. In another embodiment, the mixture can be a purified mixture. The crude mixture can include a bioorthogonally tagged protein for selective reaction with a chromatography resin. The mixture can be a lysate of cellular components. The lysate can also be a clarified lysate containing a bioorthogonally-tagged protein. The mixture can be a solubilized cellular membrane lysate. The mixture can also be a cell-free protein expression mixture. In yet another embodiment, the mixture can be any mixture comprising a bioorthogonally tagged-protein that is functionalized onto the substrate. The method can also include covalently reacting the bioorthogonally tagged protein with a click chemistry functionalized substrate via a strain-promoted azide-alkyne cylcloaddition reaction. The method can also include covalently reacting the lysate via copper catalyzed azide-alkyne cycloaddition reaction.

In yet another aspect, a method of protein enrichment is disclosed. The method can include the step of incubating a protein enrichment substrate with a buffer to produce an equilibrated mixture of substrate and buffer, further incubating the equilibrated substrate with a crude mixture containing a binding protein to produce a second mixture of functionalized substrate and crude mixture to produce a protein bound substrate, washing the protein bound substrate to produce a washed substrate, and eluting the binding protein from the substrate with another buffer to thereby result in an enriched protein. The protein enrichment substrate is prepared by a process that includes incubating a starting substrate with a mixture to produce a functionalized substrate to thereby make the protein enrichment substrate, wherein the mixture includes a bioorthogonally tagged protein for selective reaction with the substrate. The enriched protein is a purified protein.

In yet another aspect, the protein enrichment substrate can include the step of incubating a starting substrate with a purified protein mixture containing a majority of one protein of interest to produce a functionalized substrate to be used in protein enrichment or purification. In yet another aspect, the protein enrichment substrate can include the step of incubating a starting substrate with a crude protein mixture containing a number of components, only some of which are a protein of interest to produce a functionalized substrate to be used in protein enrichment or purification. It should be appreciated that for the purposes of the present disclosure, incubation is understood to refer to mixed and allowed to react for any timescales (including but not limited to seconds to hours to days).

As demonstrative of the herein described invention, we herein present a method for achieving site-specific and bioorthogonal labeling of CaM while maintaining wild-type levels of binding and activity. Calmodulin (CaM) is a widely-studied Ca²⁺-binding protein that is highly conserved across species and involved in many complex physiological processes. Previous attempts to add a reactive tag to CaM for downstream studies have either employed non-selective labeling methods or negatively impacted CaM function. It should be appreciated that we disclose herein an engineered CaM protein that undergoes site-specific and selective bioorthogonal labeling while retaining wild-type activity levels. We have also demonstrated surface capture of the engineered, labeled CaM directly from cell lysate, and we have shown that the immobilized CaM protein maintains the ability to bind a partner protein. The methods herein disclosed are readily translatable to other proteins and applications. Other proteins can include proteins that are bioorthogonally labeled; these labels can be appended to proteins in a number of ways: enzymatic attachment (as we disclosed herein) or by metabolic labeling such as site-selective non-canonical amino acid incorporation or residue selective non-canonical amino acid incorporation, or other means not explicitly stated. Other applications can include surface capture of bioorthogonally labeled protein onto solid surfaces: beads, nanoparticles, glass, plastics are examples. Further, it should be noted that any size protein (from nanoscale to macroscale), any reaction temperatures and conditions (for example, room temperature within aqueous solutions), can apply to the methods and products herein described. The reaction conditions are according to the orthogonal chemistry used for each particular case and situation.

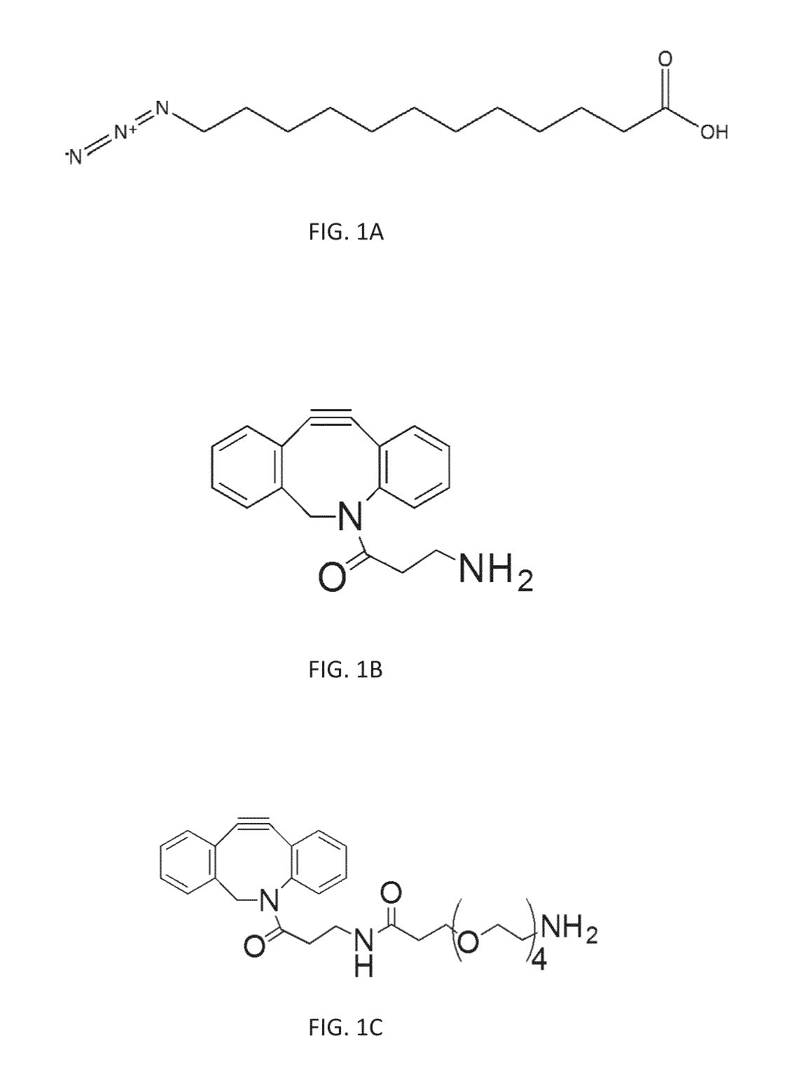

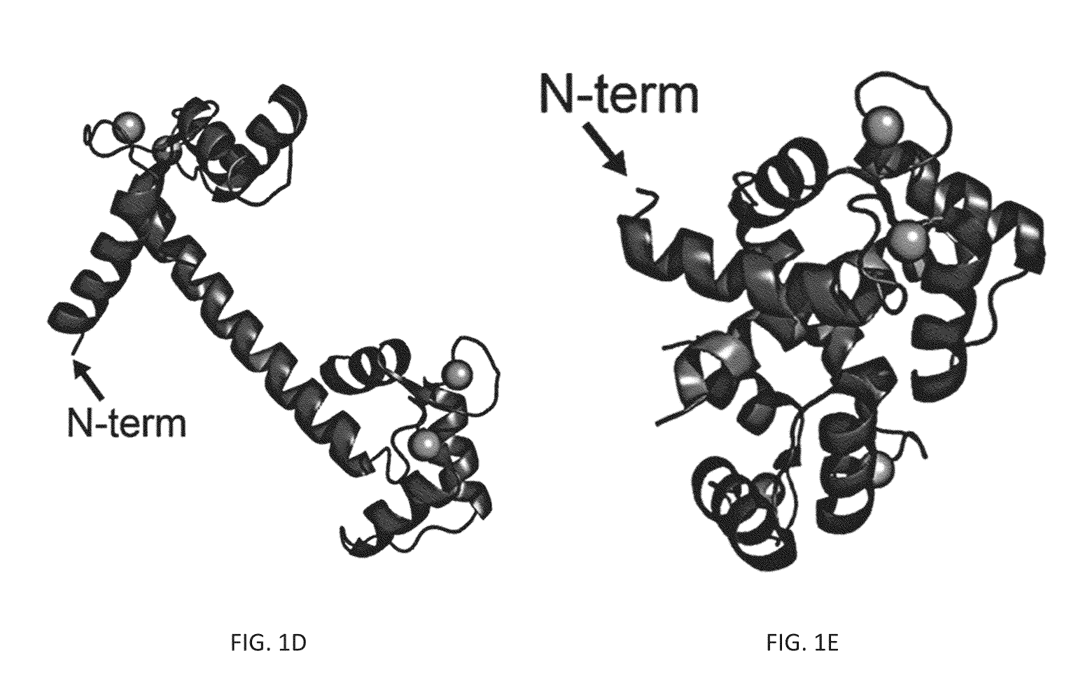

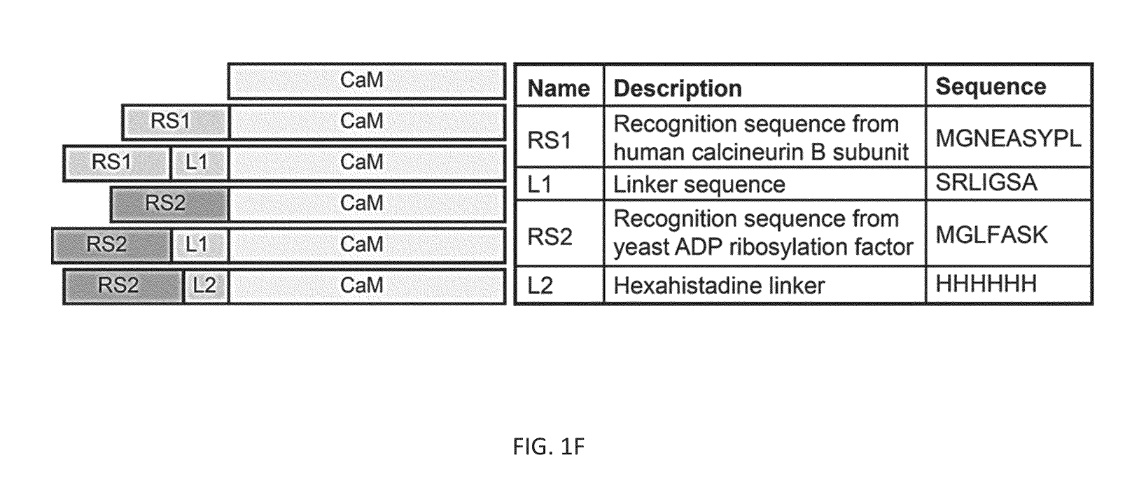

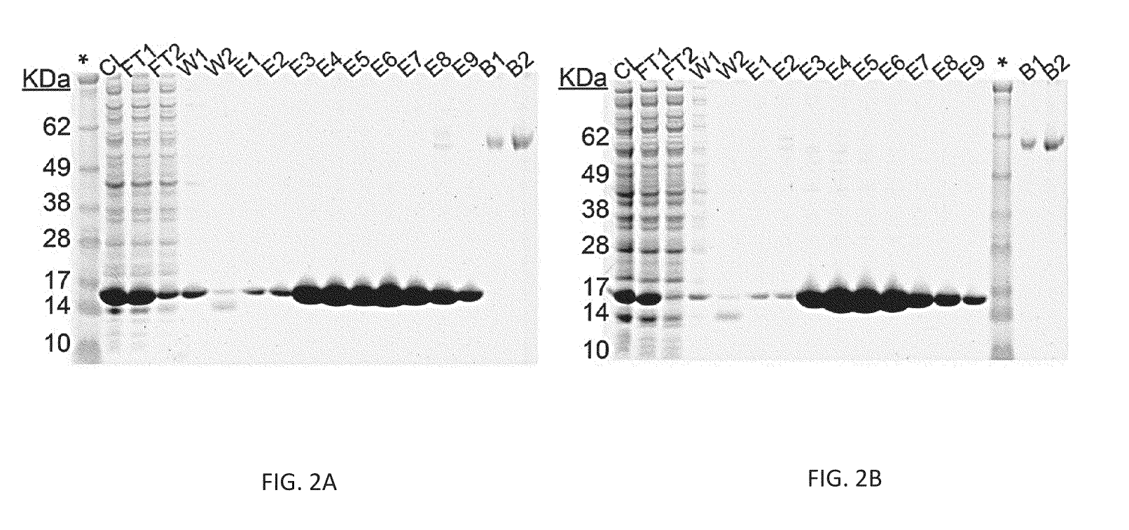

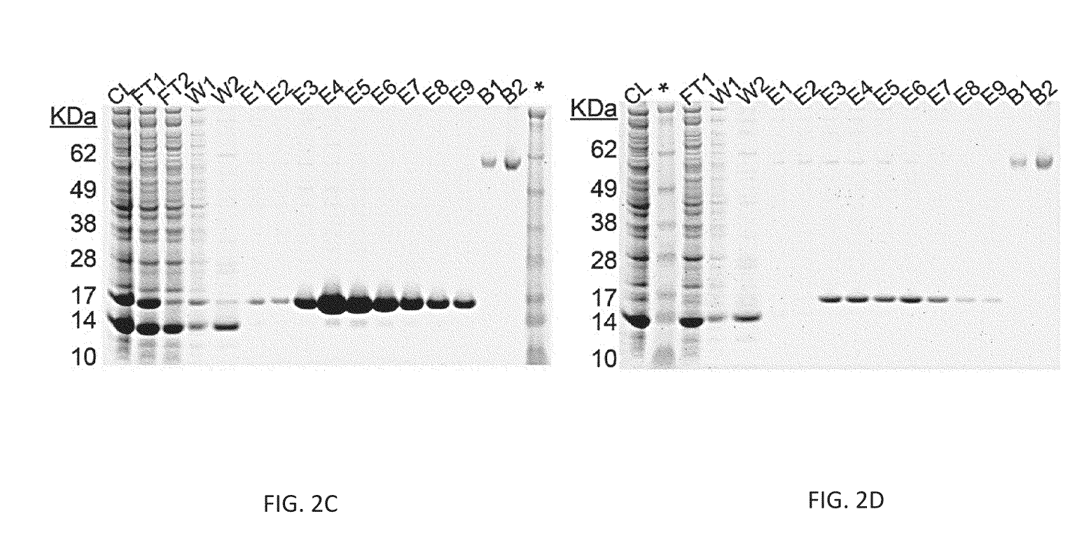

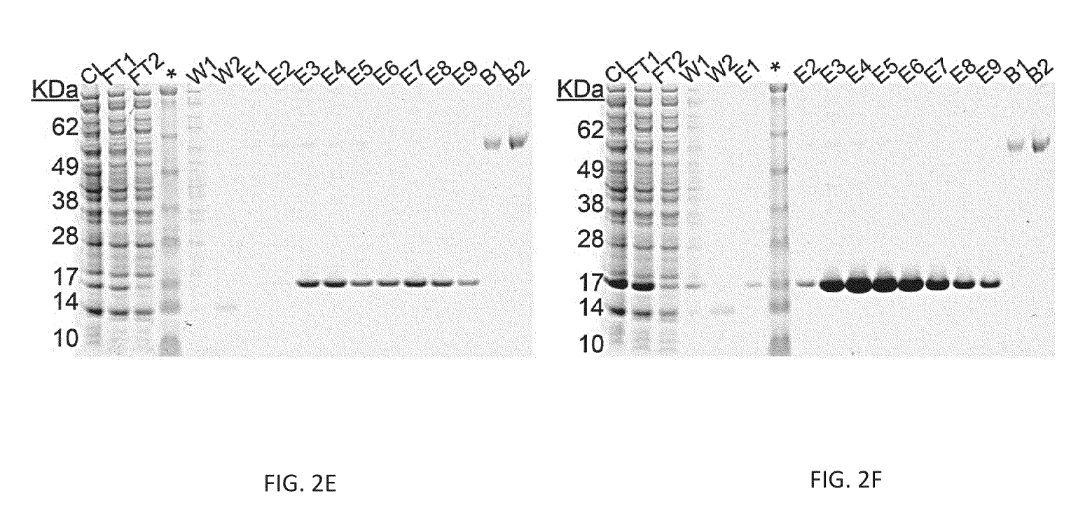

Here we describe one instance of bioorthogonal labeling of a protein of interest via one type of chemoenzymatic labeling. In a recombinant expression system, the N-myristoyl transferase (NMT) enzyme appends 12-azidododecanoic acid (ADA) (FIG. 1A), a reactive fatty acid, to the N-terminus of non-natural substrate proteins engineered to display an NMT recognition sequence. In this example, our primary objective was to engineer CaM for NMT-mediated labeling without loss of wild-type activity. We selected an NMT-based labeling strategy in part because the N-terminus of CaM appears to be solvent-accessible in both the Ca²⁺-bound and protein-bound states (FIGS. 1D and 1E). In addition, NMT-mediated protein labeling is especially well-suited for a second key objective of the current studies: surface capture of CaM for downstream applications. Because NMT is orthogonal toward bacterial proteins, surface capture of an ADA-tagged protein can be achieved directly from lysate, thus simplifying experimental protocols and potentially improving protein yields. Surface capture of purified ADA-tagged protein is also readily achieved. The promiscuity of NMT also allows for a variety of different functionalized analogs, including ADA and other bioorthogonal labels, to serve as effective substitutes for the enzyme's fatty acid substrate. Here, we report the preparation of an engineered CaM protein that is labeled site-specifically, selectively, and quantitatively by NMT with ADA with no loss of wild-type binding and activity levels. We have also developed in situ surface capture techniques for rapid generation of CaM-binding protein affinity resins that are highly effective in purifying a representative CaM-binding protein. It should be appreciated here that protein purification can be synonymous with the term protein enrichment and protein pull-down. It should also be appreciated here that resin could be understood to include any substrate (ie. glass, beads, nanoparticle, plastics, polymers, etc.). The methods and advances described here provide a promising platform for future studies of CaM; additionally, they constitute a general set of translatable techniques that should aid in the labeling and surface capture of other proteins for which maintenance of wild-type activity levels is a high priority.

Preparation and LC-MS Characterization of Engineered CaM Proteins. We designed a family of CaM proteins that were engineered to display different N-terminal amino acid sequences (FIG. 1F). An NMT recognition sequence was incorporated into each construct in order to achieve NMT-mediated protein labeling; the selected recognition sequences were derived from two well-known NMT substrate proteins, human calcineurin B (CaN) and yeast ADP-ribosylation factor. In addition, two linker sequences were chosen for inclusion between the NMT recognition sequence and CaM: a hexahistadine tag that could aid in protein purification and identification, and a linker sequence that was previously employed in a different CaM fusion protein. In total, five engineered CaM constructs were cloned and co-expressed with human NMT1 in E. coli in the presence of 12-azidododecanoic acid (ADA) (FIG. 1A). Although some variations were observed in expression levels, all five engineered proteins were successfully purified for downstream studies. Of the engineered proteins, RS1-CaM consistently resulted in the highest yield of pure protein (Table 1). Notably, the presence of the ADA label did not appear to interfere with CaM purification, which depends on hydrophobic interactions between the protein and phenyl sepharose resin. Finally, wild-type CaM (WT CaM) and the five engineered constructs were characterized by intact LC-MS; the results confirmed protein integrity and showed that all five engineered constructs were labeled with ADA by NMT in a near-quantitative manner (Table 2). The high labeling efficiency is a key factor for the success of surface capture experiments, in that nearly 100% of each engineered CaM construct is available for reaction with a “clickable” partner.

TABLE 1
Purified yields of CaM proteins.

| | Actual Yield from | Estimated Yield |
|---|---|---|
| | 100 mL Culture | from 1 L |
| Protein | (mg) | Culture (mg) |
| WT CaM | 7.3 | 73 |
| ADA-RS1-CaM | 6.2 | 62 |
| ADA-RS1-L1-CaM | 4.5 | 45 |
| ADA-R2-CaM | 0.2 | 2 |
| ADA-RS2-L1-CaM | 0.4 | 4 |
| ADA-RS2-L2-CaM | 3.5 | 35 |

TABLE 2
Engineered CaM proteins are labeled
with ADA in situ with high efficiency.

| | Expected | Observed | |
|---|---|---|---|
| | Mass | Mass | % |
| Protein | (Da)* | (Da) | Labeled^{†} |
| ADA-RS1-CaM | 17,735.02 | 17,731.59 | >98% |
| ADA-RS1-L1- | 18,419.83 | 18,416.77 | 93% |
| CaM | | | |
| ADA-RS2-CaM | 17,506.84 | 17,504.05 | 88% |
| ADA-RS2-L1- | 18,191.65 | 18,188.88 | >98% |
| CaM | | | |
| ADA-RS2-L2- | 18,329.68 | 18,327.36 | >98% |
| CaM | | | |

*All mass values account for removal of initial Met residue and addition of ADA.
^{†}Calculated from the relative abundance of peaks corresponding to labeled and unlabled forms of each protein.

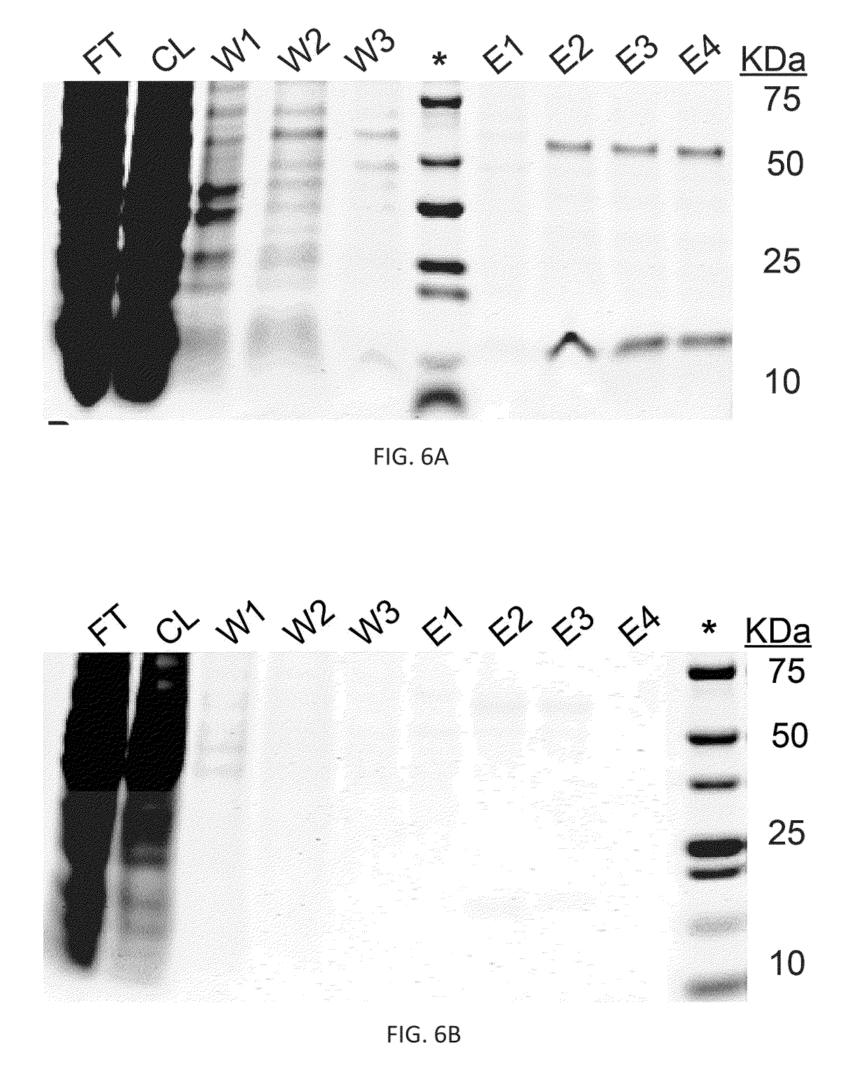

CaM-Sepharose Affinity Resins Prepared from ADA-RS1-CaM are Superior to Conventional Resins Prepared from Pure WT CaM. After establishing that ADA-RS1-CaM is highly expressed, quantitatively and selectively labeled in E. coli, and reactive towards alkyne probes, we proceeded to prepare CaM-affinity resins from ADA-RS1-CaM to exploit and showcase these features with a useful application. Protein-affinity resins are a critical component of many protein-purification protocols; for example, CaM-sepharose resin is routinely utilized for the purification of CaN, CaM kinase, and numerous other CaM-binding proteins. However, preparation of protein-affinity resins is generally labor-, time-, and resource-intensive, in part because the protein used for resin preparation must itself be purified before reaction with the resin. In contrast, we sought to prepare a protein-affinity resin directly from cell lysate (FIG. 6A). We also envisioned that the methods developed for preparation of CaM-affinity resin from ADA-RS1-CaM could be readily translated to proteins other than CaM and bioconjugation applications other than production of protein-affinity resins.

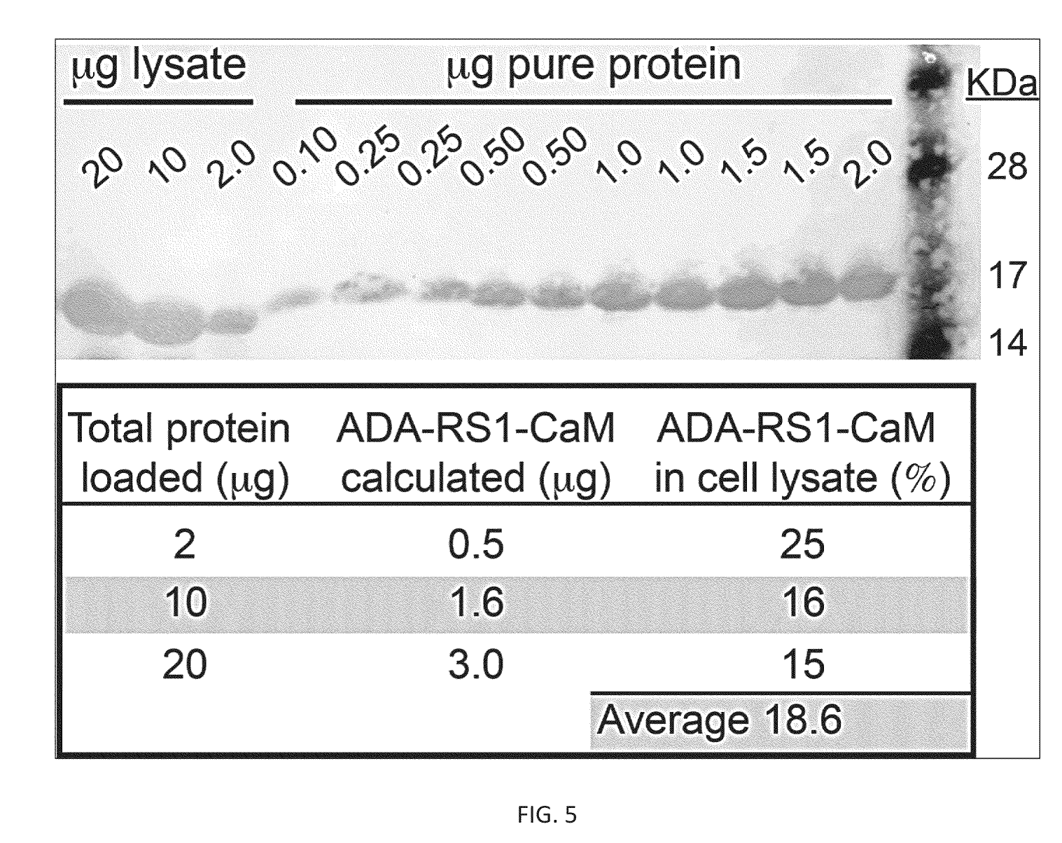

Six CaM-affinity resins were prepared and evaluated for their ability to bind and purify CaN from lysate. Two resins were produced following the conventional approach of attaching pure WT CaM via its Lys side-chains to amine-reactive sepharose beads; we selected amine-reactive resins displaying either cyanogen bromide (CnBr) or N-hydroxy succinimidyl ester (NHS) groups. In addition, four resins were produced from lysate containing CaM, rather than purified CaM: NHS-activated resin was first functionalized with ADIBO-NH2 or ADIBO-PEG-NH2 (FIG. 1B and FIG. 1C) to take advantage of the strain-promoted azide-alkyne cycloaddition reaction between ADIBO and azides, then treated with lysate containing ADA-RS1-CaM or WT CaM. It should be appreciated that although azide-alkyne cycloaddition reactions were applicable to the presently disclosed embodiment, other so-called “click” chemistries (including copper catalyzed azide-alkyne cycloaddition) can be used as well. In order to ensure that all six resins were exposed to the same quantity of CaM, whether purified or in lysate, the proportion of lysate comprised by CaM was determined in advance (FIG. 5). Finally, all six CaM-affinity resins were incubated with lysate containing CaN, which is comprised of 60-kDa and 19-kDa subunits; standard protocols were followed to elute pure CaN from each resin.

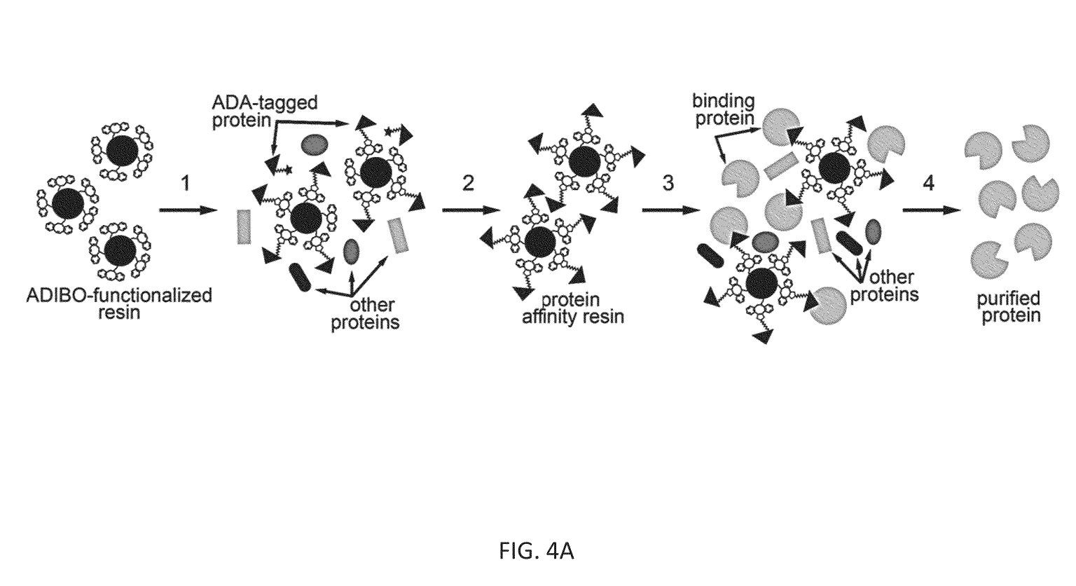

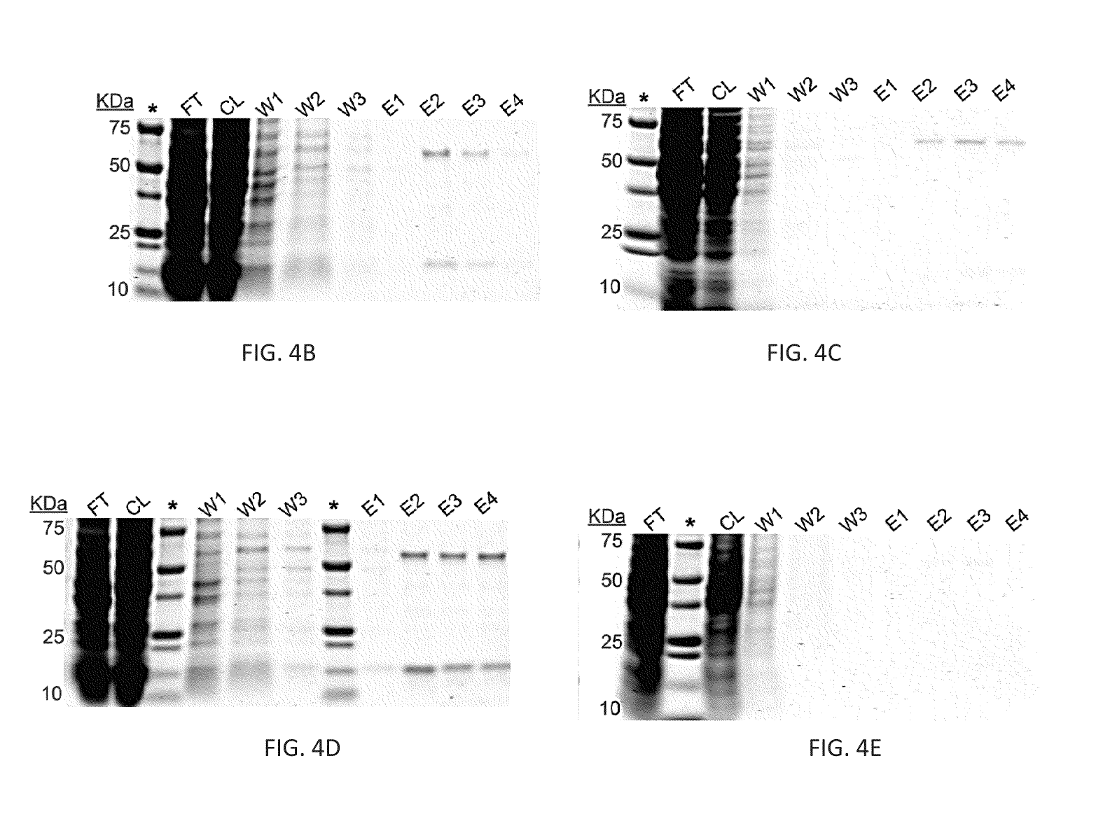

To quantify the ability of each resin to purify CaN, we analyzed the purification fractions by SDS-PAGE (FIGS. 4B-4E; FIGS. 6A and 6B). Upon Coomassie staining of the gels, it was immediately apparent that both resins prepared from lysate containing ADA-RS1-CaM were highly effective in purifying CaN (FIG. 6D; FIG. 8A). Notably, a higher yield of CaN was obtained with ADA-RS1-CaM-resin prepared from ADIBO-PEG-resin versus ADIBO-resin (Table 3). One possible explanation for the difference in yields could be that the presence of a PEG spacer element increases the distance between RS1-CaM and the resin beads, thus improving the accessibility of RS1-CaM for CaN binding. The hydrophilic PEG spacer may also decrease the occurrence of non-specific hydrophobic interactions between the RS1-CaM protein and the resin itself, also improving the probability of successful CaM/CaN binding. Both negative controls, in which resins functionalized with ADIBO-NH₂ or ADIBO-PEG-NH₂ were treated with WT CaM lysate, showed no evidence of eluted CaN (FIG. 4E; FIG. 6B); these data strongly suggest that the CaN purification results observed with ADA-RS1-CaM-resins rely on specific capture of ADA-RS1-CaM from lysate by ADIBO via the azide-alkyne cycloaddition. Finally, SDS-PAGE results showed that both resins prepared from ADA-RS1-CaM lysate were more effective for CaN purification than the two positive control resins prepared from pure WT CaM (FIGS. 4B and 4C), an exciting and unexpected result. Indeed, measurement of pure CaN yields from each resin across independent replicates revealed that, on average, a 10-fold enhancement in CaN yield was achieved when using either ADA-RS1-CaM-resin versus either WT CaM resin (Table 3).

TABLE 3
Purified CaN yields from CaM resins.

| Resin | Functionalization | CaM Source | Quantity of CaN (μg)* |
|---|---|---|---|
| CnBr | — | Pure CaM | 16.6 ± 10.8 |
| NHS | — | Pure CaM | 14.7 ± 12.8 |
| NHS | ADIBO-PEG-NH₂ | ADA-RS1-CaM | 190.0 ± 70.8 |
| | | lysate | |
| NHS | ADIBO-PEG-NH₂ | WT CaM lysate | N.D. |
| NHS | ADIBO-NH₂ | ADA-RS1-CaM | 127.1 ± 82.1 |
| | | lysate | |
| NHS | ADIBO-NH₂ | WT CaM lysate | N.D. |

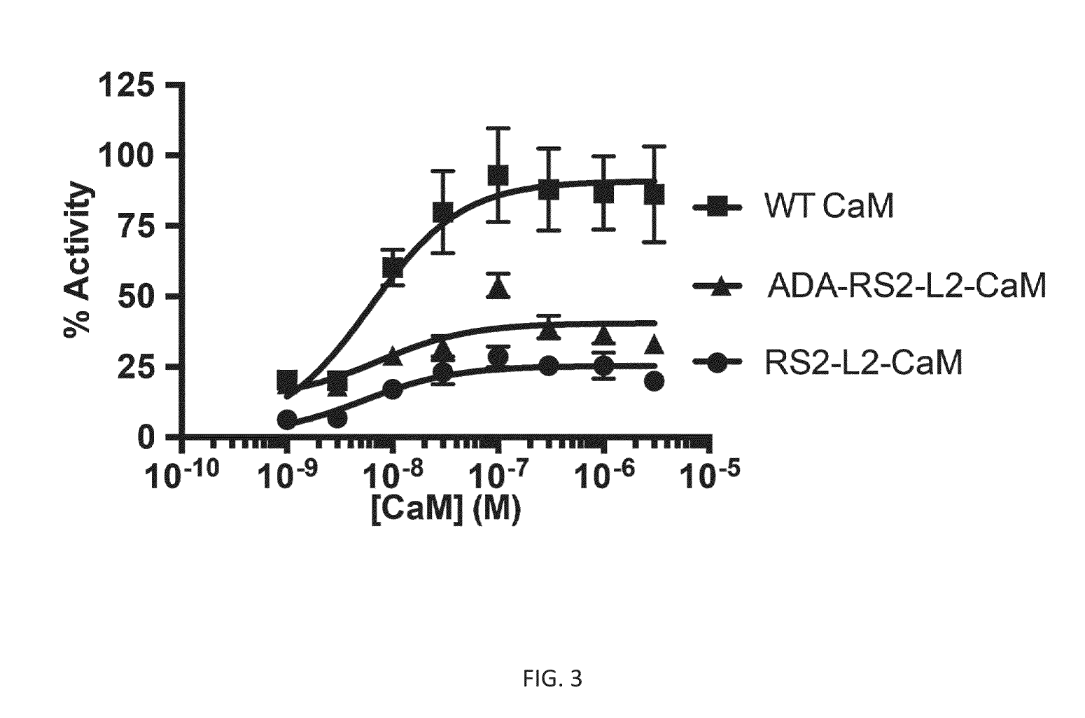

*Quantity of CaN purified from each resin in μg (mean ± SD); n = 4.
Representative SDS-PAGE gels are shown in FIG. 3.
N.D. = not detected.

Taken together, our CaM-affinity resin experiments clearly show that RS1-CaM remains active through protein engineering, labeling with ADA, and surface immobilization from lysate, thus establishing a streamlined approach to the preparation of highly effective CaM-sepharose resins for purification of CaM-binding proteins. Moreover, two key advantages of our methods are applicable to any protein engineered to display an NMT recognition sequence: (1) NMT-mediated labeling enables surface capture directly from lysate, owing to the orthogonality of NMT toward endogenous bacterial proteins, and (2) site-specific attachment of an ADA-labeled protein to a reactive partner, particularly a functionalized surface, may confer significant advantages over random attachment via primary amine chemistry or other non-specific methods. Indeed, the latter point may be partly responsible for the difference in pure CaN yields between the ADA-RS1-CaM resins and the conventionally prepared pure WT CaM resins, since primary amine chemistry gives rise to a mixture of attachment points and, as a result, a variety of protein orientations on the resin. Most importantly, our work lays the foundation for the purification of other CaM-binding proteins with ADA-RS1-CaM resins, the production of other protein resins following our methods, and the development of other in-lysate bioconjugation applications employing ADA-labeled proteins. In particular, the methods described herein can be especially useful for applications in which the functionality of a protein must be maintained through labeling and conjugation, and for proteins which are difficult to purify from lysates.

### EXAMPLE 1

### Preparation of CaM-Affinity Resins

Cloning, Protein Expression, and Protein Labeling. The template plasmid for all engineered CaM constructs was pET-15b encoding Drosophila melanogaster wild-type CaM. Primers encoding the NMT recognition sequences and linker sequences were used in conjunction with the QuikChange site-directed mutagenesis kit to prepare plasmids encoding the engineered CaM proteins, following the Two-Step PCR method. Plasmids were transformed into chemically competent BL21(DE3) cells already containing a plasmid encoding human NMT1 and MetAP. Expression cultures were grown in an incubator/shaker (37° C., 250 rpm) in LB medium supplemented with 50 μg/mL kanamycin and 100 μg/mL ampicillin. Once cultures grew to an OD600 of 0.8-1.0, protein expression was induced with 1 mM IPTG, and 500 μM ADA (compound from FIG. 1A, from a 500 mM stock in DMSO) was added simultaneously. After 3-4 hr of protein expression, cells were harvested via centrifugation. Cell pellets were washed with cold PBS and stored at −80° C. until use.

Preparation of CaM-Affinity Resins. CnBr-Sepharose and NHS-Sepharose were prepared for primary amine reaction according to the manufacturer's instructions. Pure CaM resins were prepared according to previously published methods: briefly, one column volume of 0.5 mg/mL purified porcine brain CaM in 50 mM HEPES (pH 7.5) was incubated with either CnBr-activated resin or NHS-activated resin for 2-3 hr at 4° C. on a rotator. To prepare ADIBO- and ADIBO-PEG-functionalized resins, one column volume of 12.5 mM ADIBO-NH2 or ADIBO-PEG-NH2 (compounds in FIGS. 1B or 1C1) in 50% DMSO and 10 mM HEPES (pH 7.5) was added to the resin. All resins were deactivated with four column volumes of 1 M Tris (pH 8.0) for 1 hr. ADIBO- and ADIBO-PEG-resins were equilibrated for CaM coupling with four column volumes of 50 mM Tris (pH 7.5). Frozen cell pellets from 100 mL E. coli cultures expressing ADA-RS1-CaM or WT CaM were lysed in CaM Lysis Buffer (50 mM Tris (pH 7.5), 10 mM KCl, 0.5 mM EGTA, 0.5 mM EDTA, 1 mg/mL lysozyme, 10 units DNase) via sonication, then clarified via centrifugation. The total protein concentration of each clarified lysate was quantified using a Pierce 660 nm Protein Assay. One column volume of clarified lysate at 3.5 mg/mL total protein concentration (0.5 mg/mL estimated CaM concentration) was incubated with ADIBO- or ADIBO-PEG-resin overnight at 4° C. on a rotator. CaM-conjugated resins were washed ×3 with 10 column volumes of the following wash buffers: Wash 1 (100 mM ammonium carbonate (pH 8.6), 2 mM EGTA), Wash 2 (10 mM Tris (pH 7.5), 1 M NaCl, 2 mM CaCl₂), and Wash 3 (100 mM sodium acetate (pH 4.6), 2 mM CaCl₂). The resins were either stored in 20% ethanol or immediately equilibrated for CaN purification with Buffer A (25 mM Tris (pH 7.5), 2 mM CaCl₂, 0.1 mM EDTA, 0.5 mM DTT).

### EXAMPLE 2

### Purification of Calcineurin

Calcineurin (CaN) purification was performed according to previously published methods for CaM-Sepharose purification of CaN. Briefly, a frozen cell pellet from a 500 mL E. coli culture expressing CaN was lysed in CaN Lysis Buffer (25 mM Tris (pH 7.5), 3 mM MgCl₂, 100 mM NaCl, 2 mM CaCl₂, 0.5 mM PMSF, Roche Complete Protease Inhibitor Cocktail, and 1 mg/mL lysozyme). Clarified lysate (2 mL) and 1 mL of resin equilibrated with Buffer A were incubated for 30 min at 4° C. on a rotator. It should be appreciated that clarified lysate represents only one embodiment of the present disclosure. Other click chemistry samples may be used as well (for example, non-clarified lysate, solubilized-membrane fractions, or cell-free protein expressions can be used). Resins were washed sequentially with Buffer A, Buffer A plus 1 M NaCl, and Buffer A. During the final wash, resins were transferred to BioRad MicroSpin columns that were pre-washed with CaN Lysis Buffer. Elution was accomplished with sequential additions of 0.5 mL Elution Buffer (25 mM Tris (pH 7.5), 3 mM MgSO₄, 1 mM EGTA, 0.5 mM DTT). Elution fractions were analyzed via SDS-PAGE and pooled. Quantification: The concentration of CaN in the pooled elution sample from each resin was quantified via SDS-PAGE with Coomassie staining. Resin elution samples and a calibration curve of BSA (66 kDa) and lysozyme (14 kDa) were run in duplicate on a protein gel. Integrated band intensities from each gel were obtained with Li-Cor Odyssey Software. Concentrations of the CaN-A subunit (60 kDa) and the CaN-B subunit (19 kDa) were interpolated from the BSA and lysozyme standard curves, respectively, then added together to give the concentration of total CaN purified. Finally, the quantity of CaN purified was calculated as the concentration multiplied by the pooled elution volume.

### EXAMPLE 3

### Kits for Facilitating Enrichment

An example embodiment and implementation of the herein described methods and products can include a kit wherein the kit includes a protein enrichment substrate and buffers. The protein enrichment substrate can include a bioorthogonally labeled protein of choice attached to a starting substrate using the methods described herein. Buffers can include solutions configured to facilitate protein binding to the protein enrichment substrate, washing of the substrate, release of enriched proteins from the substrate, and solutions for storage of the substrate.

### EXAMPLE 4

### Kits for Facilitating Protein Expression

As another example embodiment of the implementation of the herein described methods and products, the kit presented in Example 3 can also optionally include genes of choice so that the protein to be purified can be expressed. This kit can be used to facilitate the purification over the herein described resins.

### EXAMPLE 5

### Methods and Kits for User-Defined Protein Enrichment

As yet another example embodiment for implementing the herein described methods and products, a user can be given the option to bioorthogonally label the protein of the user's choice, by dictating the gene sequence the user desires. Accordingly, a protein enrichment substrate can be made to fit the user's needs using the herein described methods. The protein enrichment can include protein purification.

Those skilled in the art will recognize that numerous modifications can be made to the specific implementations described above. The implementations should not be limited to the particular limitations described. Other implementations may be possible. In addition, all references cited herein are indicative of the level of skill in the art and are hereby incorporated by reference in their entirety.

### REFERENCES

1. Friedberg F & Rhoads A R (2001) Evolutionary aspects of calmodulin. IUBMB life 51(4):215-221.
2. Swan D G, Hale R S, Dhillon N, & Leadlay P F (1987) A bacterial calcium-binding protein homologous to calmodulin. Nature 329(6134):84-85.
3. Toutenhoofd S L & Strehler E E (2000) The calmodulin multigene family as a unique case of genetic redundancy: multiple levels of regulation to provide spatial and temporal control of calmodulin pools? Cell Calcium 28(2):83-96.
4. Means A R & Rasmussen C D (1988) Calcium, calmodulin and cell proliferation. Cell Calcium 9(5-6):313-319.
5. Ui-Tei K, Nagano M, Sato S, & Miyata Y (2000) Calmodulin-dependent and -independent apoptosis in cell of a Drosophila neuronal cell line. Apoptosis: an international journal on programmed cell death 5(2): 133-140.
6. Maier L S & Bers D M (2002) Calcium, calmodulin, and calcium-calmodulin kinase II: heartbeat to heartbeat and beyond. J. Mol. Cell. Cardiol. 34(8):919-939.
7. Saucerman J J & Bers D M (2012) Calmodulin binding proteins provide domains of local Ca2+ signaling in cardiac myocytes. J. Mol. Cell. Cardiol. 52(2):312-316.
8. Pang Z P, Cao P, Xu W, & Sudhof T C (2010) Calmodulin controls synaptic strength via presynaptic activation of calmodulin kinase II. The Journal of neuroscience: the official journal of the Society for Neuroscience 30(11):4132-4142.
9. Xia Z & Storm D R (2005) The role of calmodulin as a signal integrator for synaptic plasticity. Nat Rev Neurosci 6(4):267-276.
10. Chin D & Means A R (2000) Calmodulin: a prototypical calcium sensor. Trends Cell Biol. 10(8):322-328.
11. Dadi P K, et al. (2014) Inhibition of pancreatic beta-cell Ca2+/calmodulin-dependent protein kinase II reduces glucose-stimulated calcium influx and insulin secretion, impairing glucose tolerance. The Journal of biological chemistry 289(18):12435-12445.
12. Zou J, et al. (2014) Gap junction regulation by calmodulin. FEBS Lett. 588(8):1430-1438.
13. Berchtold M W & Villalobo A (2014) The many faces of calmodulin in cell proliferation, programmed cell death, autophagy, and cancer. Biochim. Biophys. Acta 1843(2):398-435.
14. Olwin B B & Storm D R (1983) Preparation of fluorescent labeled calmodulins. Methods in enzymology 102:148-157.
15. Torok K, et al. (1998) Inhibition of calmodulin-activated smooth-muscle myosin light-chain kinase by calmodulin-binding peptides and fluorescent (phosphodiesterase-activating) calmodulin derivatives. Biochemistry 37(17):6188-6198.
16. Torok K & Trentham D R (1994) Mechanism of 2-chloro-(epsilon-amino-Lys75)-[6-[4-(N,N-diethylamino)phenyl]-1,3,5-triazin-4-yl]calmodulin interactions with smooth muscle myosin light chain kinase and derived peptides. Biochemistry 33(43):12807-12820.
17. Waxham M N, Tsai A L, & Putkey J A (1998) A mechanism for calmodulin (CaM) trapping by CaM-kinase II defined by a family of CaM-binding peptides. The Journal of biological chemistry 273(28):17579-17584.
18. Quintana A R, Wang D, Forbes J E, & Waxham M N (2005) Kinetics of calmodulin binding to calcineurin. Biochem. Biophys. Res. Commun. 334(2):674-680.
19. Meyer T, Hanson P I, Stryer L, & Schulman H (1992) Calmodulin Trapping by Calcium-Calmodulin-Dependent Protein Kinase. Science 256(5060): 1199-1202.
20. Mills J S, Walsh M P, Nemcek K, & Johnson J D (1988) Biologically active fluorescent derivatives of spinach calmodulin that report calmodulin target protein binding. Biochemistry 27(3):991-996.
21. Dedman J R, Potter J D, Jackson R L, Johnson J D, & Means A R (1977) Physicochemical properties of rat testis Ca2+-dependent regulator protein of cyclic nucleotide phosphodiesterase. Relationship of Ca2+-binding, conformational changes, and phosphodiesterase activity. The Journal of biological chemistry 252(23):8415-8422.
22. Kulkarni C, Kinzer-Ursem T L, & Tirrell D A (2013) Selective functionalization of the protein N terminus with N-myristoyl transferase for bioconjugation in cell lysate. Chembiochem 14(15):1958-1962.
23. Duronio R J, et al. (1990) Protein N-myristoylation in Escherichia coli: reconstitution of a eukaryotic protein modification in bacteria. Proceedings of the National Academy of Sciences of the United States of America 87(4):1506-1510.
24. Lu T, et al. (1994) The substrate specificity of Saccharomyces cerevisiae myristoyl-CoA: protein N-myristoyltransferase. Polar probes of the enzyme's myristoyl-CoA recognition site. The Journal of biological chemistry 269(7):5346-5357.
25. Devadas B, et al. (1992) Substrate specificity of Saccharomyces cerevisiae myristoyl-CoA: protein N-myristoyltransferase. Analysis of fatty acid analogs containing carbonyl groups, nitrogen heteroatoms, and nitrogen heterocycles in an in vitro enzyme assay and subsequent identification of inhibitors of human immunodeficiency virus I replication. The Journal of biological chemistry 267(11):7224-7239.
26. Kishore N S, et al. (1991) The substrate specificity of Saccharomyces cerevisiae myristoyl-CoA:protein N-myristoyltransferase. Analysis of myristic acid analogs containing oxygen, sulfur, double bonds, triple bonds, and/or an aromatic residue. The Journal of biological chemistry 266(14):8835-8855.
27. Boutin J A (1997) Myristoylation. Cellular signalling 9(1):15-35.
28. Kennedy M T, Brockman H, & Rusnak F (1996) Contributions of myristoylation to calcineurin structure/function. The Journal of biological chemistry 271(43):26517-26521.
29. Van Valkenburgh H A & Kahn R A (2002) Coexpression of proteins with methionine aminopeptidase and/or N-myristoyltransferase in Escherichia coli to increase acylation and homogeneity of protein preparations. Methods in enzymology 344:186-193.
30. Li C J, et al. (1999) Dynamic redistribution of calmodulin in HeLa cells during cell division as revealed by a GFP-calmodulin fusion protein technique. J. Cell Sci. 112 (Pt 10):1567-1577.
31. Rhyner J A, Koller M, Durussel-Gerber I, Cox J A, & Strehler E E (1992) Characterization of the human calmodulin-like protein expressed in Escherichia coli. Biochemistry 31(51):12826-12832.
32. Stemmer P M & Klee C B (1994) Dual calcium ion regulation of calcineurin by calmodulin and calcineurin B. Biochemistry 33(22):6859-6866.
33. Shifman J M & Mayo S L (2002) Modulating calmodulin binding specificity through computational protein design. J. Mol. Biol. 323(3):417-423.
34. Tornoe C W, Christensen C, & Meldal M (2002) Peptidotriazoles on solid phase: [1,2,3]-triazoles by regiospecific copper(i)-catalyzed 1,3-dipolar cycloadditions of terminal alkynes to azides. J. Org. Chem. 67(9):3057-3064.
35. Rostovtsev V V, Green L G, Fokin V V, & Sharpless K B (2002) A stepwise huisgen cycloaddition process: copper(I)-catalyzed regioselective “ligation” of azides and terminal alkynes. Angewandte Chemie (International ed. in English) 41(14):2596-2599.
36. Wang Q, et al. (2003) Bioconjugation by copper(I)-catalyzed azide-alkyne [3+2] cycloaddition. Journal of the American Chemical Society 125(11):3192-3193.
37. Lukas T J & Watterson D M (1988) Purification of Calmodulin and Preparation of Immobilized Calmodulin. Methods Enzymol. 157:328-339.
38. Westcott K R, La Porte D C, & Storm D R (1979) Resolution of adenylate cyclase sensitive and insensitive to Ca2+ and calcium-dependent regulatory protein (CDR) by CDR-sepharose affinity chromatography. Proceedings of the National Academy of Sciences of the United States of America 76(1):204-208.
39. Agard N J, Prescher J A, & Bertozzi C R (2004) A strain-promoted [3+2] azide-alkyne cycloaddition for covalent modification of biomolecules in living systems. Journal of the American Chemical Society 126(46):15046-15047.
40. Kuzmin A, Poloukhtine A, Wolfert M A, & Popik V V (2010) Surface functionalization using catalyst-free azide-alkyne cycloaddition. Bioconjugate chemistry 21(11):2076-2085.
41. Mondragon A, et al. (1997) Overexpression and purification of human calcineurin alpha from Escherichia coli and assessment of catalytic functions of residues surrounding the binuclear metal center. Biochemistry 36(16):4934-4942.
42. Chattopadhyaya R, Meador W E, Means A R, & Quiocho F A (1992) Calmodulin structure refined at 1.7 A resolution. J. Mol. Biol. 228(4):1177-1192.
43. Dunlap T B, et al. (2014) Stoichiometry of the calcineurin regulatory domain-calmodulin complex. Biochemistry 53(36):5779-5790.
44. Shifman J M & Mayo S L (2003) Exploring the origins of binding specificity through the computational redesign of calmodulin. Proceedings of the National Academy of Sciences of the United States of America 100(23): 13274-13279.
45. Wang W & Malcolm B A (1999) Two-Stage PCR Protocol Allowing Introduction of Multiple Mutations, Deletions and Insertions Using QuikChange™ Site-Directed Mutagenesis. BioTechniques 26:680-682.
46. Perrino B A, Ng L Y, & Soderling T R (1995) Calcium regulation of calcineurin phosphatase activity by its B subunit and calmodulin. Role of the autoinhibitory domain. The Journal of biological chemistry 270(1):340-346.
47. Shifman J M, Choi M H, Mihalas S, Mayo S L, & Kennedy M B (2006) Ca2+/calmodulin-dependent protein kinase II (CaMKII) is activated by calmodulin with two bound calciums. Proceedings of the National Academy of Sciences of the United States of America 103(38):13968-13973.
48. Gaertner T R, et al. (2004) Comparative analyses of the three-dimensional structures and enzymatic properties of alpha, beta, gamma and delta isoforms of Ca2+-calmodulin-dependent protein kinase II. The Journal of biological chemistry 279(13):12484-12494.
49. Bers D M, Patton C W, & Nuccitelli R (2010) A practical guide to the preparation of Ca(2+) buffers. Methods in cell biology 99:1-26.
50. Perrino B A, Ng L Y, & Soderling T R (1995) Calcium regulation of calcineurin phosphatase activity by its B subunit and calmodulin. Role of the autoinhibitory domain. The Journal of biological chemistry 270(1):340-346.
51. Stemmer P M & Klee C B (1994) Dual calcium ion regulation of calcineurin by calmodulin and calcineurin B. Biochemistry 33(22):6859-6866.
52. Shifman J M, Choi M H, Mihalas S, Mayo S L, & Kennedy M B (2006) Ca2+/calmodulin-dependent protein kinase II (CaMKII) is activated by calmodulin with two bound calciums. Proceedings of the National Academy of Sciences of the United States of America 103(38):13968-13973.
53. Shifman J M & Mayo S L (2002) Modulating calmodulin binding specificity through computational protein design. J. Mol. Biol. 323(3):417-423.
54. Mondragon A, et al. (1997) Overexpression and purification of human calcineurin alpha from Escherichia coli and assessment of catalytic functions of residues surrounding the binuclear metal center. Biochemistry 36(16):4934-4942.
55. Van Valkenburgh H A & Kahn R A (2002) Coexpression of proteins with methionine aminopeptidase and/or N-myristoyltransferase in Escherichia coli to increase acylation and homogeneity of protein preparations. Methods in enzymology 344:186-193.
56. Devadas B, et al. (1992) Substrate specificity of Saccharomyces cerevisiae myristoyl-CoA: protein N-myristoyltransferase. Analysis of fatty acid analogs containing carbonyl groups, nitrogen heteroatoms, and nitrogen heterocycles in an in vitro enzyme assay and subsequent identification of inhibitors of human immunodeficiency virus I replication. The Journal of biological chemistry 267(11):7224-7239.
57. Kulkarni C, Kinzer-Ursem TL, & Tirrell DA (2013) Selective functionalization of the protein N terminus with N-myristoyl transferase for bioconjugation in cell lysate. Chembiochem 14(15):1958-1962.
58. Gaertner T R, et al. (2004) Comparative analyses of the three-dimensional structures and enzymatic properties of alpha, beta, gamma and delta isoforms of Ca2+-calmodulin-dependent protein kinase II. The Journal of biological chemistry 279(13):12484-12494.
59. Bers D M, Patton C W, & Nuccitelli R (2010) A practical guide to the preparation of Ca(2+) buffers. Methods in cell biology 99:1-26.
60. Rhyner J A, Koller M, Durussel-Gerber I, Cox J A, & Strehler E E (1992) Characterization of the human calmodulin-like protein expressed in Escherichia coli. Biochemistry 31(51):12826-12832.
61. Li C J, et al. (1999) Dynamic redistribution of calmodulin in HeLa cells during cell division as revealed by a GFP-calmodulin fusion protein technique. J. Cell Sci. 112 (Pt 10):1567-1577.

1. A protein enrichment substrate prepared by a process comprising incubating a starting substrate with a mixture to produce a functionalized substrate to thereby make the protein enrichment substrate, wherein the mixture comprises a bioorthogonally tagged protein for selective reaction with the substrate.

2. The protein enrichment substrate of claim 1, wherein the starting substrate is configured to be reactive to bioorthogonally labeled proteins.

3. The protein enrichment substrate of claim 2, wherein the mixture is a crude mixture.

4. The protein enrichment substrate of claim 2, wherein the mixture is a purified protein mixture.

5. The protein enrichment substrate of claim 2, wherein the starting substrate comprises magnetic beads.

6. The protein enrichment substrate of claim 2, wherein the starting substrate comprises polystyrene beads.

7. The protein enrichment substrate of claim 2, wherein the starting substrate comprises nanoparticles.

8. The protein enrichment substrate of claim 2, wherein the starting substrate comprises a chromatography resin.

9. The protein enrichment substrate of claim 2, wherein the starting substrate comprises silica oxide.

10. The protein enrichment substrate of claim 2, wherein the starting substrate comprises a polymer.

11. The polymer of claim 10, wherein the polymer is a polymer matrix.

12. A method for making an protein enrichment substrate, comprising:
incubating a substrate with a mixture to produce a functionalized substrate to thereby make the protein enrichment substrate.

13. The method of claim 12, wherein the mixture is a crude mixture.

14. The method of claim 12, wherein the mixture is a purified mixture.

15. The method of claim 13, wherein the crude mixture comprises a bioorthogonally tagged protein for selective reaction with a chromatography resin.

16. The method of claim 12, wherein the mixture is a lysate of cellular components.

17. The method of claim 16, wherein the lysate is a clarified lysate containing bioorthogonal-tagged protein.

18. The method of claim 12, wherein the mixture is a solubilized cellular membrane lysate.

19. The method of claim 12, wherein the mixture is a cell-free protein expression mixture

20. The method of claim 12, wherein the mixture is any mixture comprising a bioorthogonally tagged-protein that is functionalized onto the substrate.

21. The method of claim 20 further comprising covalently reacting the bioorthogonally tagged protein with a click chemistry functionalized substrate via a strain-promoted azide-alkyne cylcloaddition reaction.

22. The method of claim 20, further comprising covalently reacting the lysate via copper catalyzed azide-alkyne cycloaddition reaction.

23. A method of protein enrichment, comprising
incubating a protein enrichment substrate with a buffer to produce an equilibrated mixture of substrate and buffer;
further incubating the equilibrated substrate with a crude mixture containing a binding protein to produce a second mixture of functionalized substrate and crude mixture to produce a protein bound substrate;
washing the protein bound substrate to produce a washed substrate; and
eluting the binding protein from the substrate with another buffer to thereby result in an enriched protein.

24. The method of claim 23, wherein the protein enrichment substrate is prepared by a process comprising the steps of:
incubating a starting substrate with a mixture to produce a functionalized substrate to thereby make the protein enrichment substrate, wherein the mixture comprises a bioorthogonally tagged protein for selective reaction with the substrate.

25. The method of claim 23, wherein the enriched protein is a purified protein.
